(19) United States
(12) Patent Application Publication
(10) Pub. No.: US 2014/0062717 A1
(43) Pub. Date: Mar. 6, 2014

(54) Method for RFID Communication Using Inductive Orthogonal Coupling For Wireless Medical Implanted Sensors and Other Short-Range Communication Applications

(71) Applicant: The University of Iowa Research Foundation, Iowa City, IA (US)

(72) Inventors: Raghuraman Mudumbai, Coralville, IA (US); Josiah McClurg, North Liberty, IL (US); Hema K. Achanta, Iowa City, IA (US); John N. Pienta, Iowa City, IA (US); Soura Dasgupta, Coralville, IA (US); Christopher Buresh, Coralville, IA (US); Gerene Denning, Iowa City, IA (US)

(73) Assignee: The University of Iowa Research Foundation, Iowa City, IA (US)

(21) Appl. No.: 14/015,386

(22) Filed: Aug. 30, 2013

Related U.S. Application Data
(60) Provisional application No. 61/695,840, filed on Aug. 31, 2012.

(51) Int. Cl. A61B 5/00; G06K 7/10; G06K 19/07

(52) U.S. Cl. CPC A61B 5/0015; G06K 19/0717; G06K 7/10366
USPC 340/870.01; 235/492; 235/439

(57) ABSTRACT

The description provides a signal detection system employing a wireless, passive detection device that utilizes waveform shifting for reporting signals to a reader device. The system is useful for a variety of applications including as an implanted medical device for monitoring patient conditions.

The present application claims benefit of priority to U.S. Provisional Application Ser. No. 61/695,840, filed Aug. 31, 2012, the entire contents of which are hereby incorporated by reference.

### BACKGROUND OF THE INVENTION

I. Field of the Invention

The invention relates to systems and methods for detecting biologic parameters in a subject, and more particularly to an implantable device system that employs passive transmission and a unique signal transduction process to report in vivo conditions to medical personnel. Specific embodiments include intracranial implantation to measure pressure of cerebral-spinal fluid, blood sugar level monitors, pH sensors and tissue oxygen saturation detectors.

II. Background of the Invention

Radio frequency can be used to transmit information from a transmitter to a receiver. This messaging system can and has taken many formats. Recent advancements in the field have occurred in the short distance transfer of information, resulting in advancements such as radio frequency identification (RFID). Short distance wireless communication has been used in diverse applications from feed animal organization to low-pressure tire sensors. In particular, implantable medical devices have taken advantages of this type of technology, thereby permitting accurate monitoring of in vivo conditions while requiring only a single invasive procedure.

The transmitters of these types of devices are divided into three categories: active, semi-passive and passive. Self-powered (battery requiring) active transmitters are capable of transmitting an information carrying signal at any time without any intervention from an external device, semi-passive transmitters also power their own signal, but only transmit it when they receive a triggering signal from a nearby receiver. In contrast, a (batteryless) passive transmitter is unable to transmit unless it receives a powering signal from a nearby receiver.

Current passive RFID devices have a reader coil and a tag coil that communicate and transfer data through a technique commonly called backscatter modulation. When the reader is in the vicinity of the tag, the electromagnetic signal transmitted by the reader coil (antenna) powers up the tag coil (antenna). The tag uses the same signal to send back information to the reader by modulating the reader carrier signal, usually by causing a small sequence of changes in the amplitude, frequency or phase of the carrier signal. The effect of the modulation on the reader coil is typically very small (usually 50 to 100 times weaker) as compared to the carrier wave, and therefore requires a highly sensitive detector which needs to separate the modulated signal buried underneath the much stronger carrier signal. This type of backscatter modulation requires the tag coil to be within the near-field of the reader coil; this in turn means that lower frequencies (˜10 MHz) are better suited for passive RFID systems.

Active and semi-passive tags on the other hand, do not require the tags to be in the near-field of the receiver and therefore often use higher RF frequencies (>1 GHz). At these frequencies, much wider bandwidths are available, however there is also much higher levels of interference.

Passive devices can be simpler, less expensive, longer-lasting (especially because they do not need batteries) and smaller than active and semi-passive devices. Active and semi-passive tags are capable of continuously monitoring and recording information and transmitting arbitrary types and quantities of information encoded into a sequence of bits. Passive tags are limited to relatively small number of bits which must be generated or updated in real-time upon energization of the tag by a receiver. The current invention is especially well-suited for applications involving monitoring of slowly-varying biological or physical signals such as inter-cranial pressure or blood sugar levels. These signals can be assumed to be constant over the short-time intervals (several seconds) over which the measurements are taken. Therefore, extensive amounts of filtering and averaging can be applied to substantially remove noise from the measurement. Furthermore and most crucially, instead of attempting to detect minute amplitude variations from underneath a strong carrier signal, we can use the shape of the backscattered signal for a more accurate and sensitive detection. Specifically, if the tag includes a non-linear and/or time-varying element, harmonics will be introduced into the backscattered signal that were not present in the original carrier signal itself. Thus, using simple frequency domain techniques, the strong carrier signal can be easily and effectively isolated from the tag-dependent back-scatter signal. By optimally combining the amplitude and phase of the various significant harmonics in the back-scatter signal, it is possible to construct a very accurate and sensitive estimate of the sensed signal.

For example, the authors of [1] use microwave wireless link between the transmitter and the receiver to transmit signals. Their design captures the changes in the sensor output using a Voltage Controlled Oscillator. Most importantly, their design uses a battery supply on the implant device thereby making it a non-passive implant device.

There has been work in implantable wireless medical devices. The FDA approved Verichip tag being one of them [2]. This tag was designed for the purpose of patient identification. The patent [3] describes a diffraction based data carrier. This becomes particularly hard to implement for implantable medical devices.

The current RFID tag designs rely on envelope detection method for extracting data from the tag signal [4]. However, in implanted medical device settings, the sensors produce very small amplitude fluctuations in the envelope thereby making it hard to achieve high levels of accuracy.

The patent [5] describes RFID tag design that uses digital devices to provide for memory and for processing. The tag signal is first filtered to remove the carrier signal, which removes the harmonics.

Each of these examples exhibits one or more of the limitations discussed above. Therefore, improved systems for the transmission of signals from implanted devices are needed.

### SUMMARY OF THE INVENTION

Accordingly, the present invention provides for an implantable device and system for detecting and reporting biological parameters in a subject is provided. More particularly, the invention provides a passive transmitter tag device suitable for implantation into a subject, said device comprising (a) a non-linear impedance element or time-varying element for introducing harmonics or frequency-shifted signal components into a back-emf produced by a sinusoidal carrier signal; (b) an energy harnessing element; (c) sensor/transducer for monitoring a patient condition, wherein said sensor is operably connected to said non-linear impedance element or time-varying element. The sensor/transducer may be a pressure monitor, a pH monitor, a temperature monitor, or a blood glucose monitor. The energy harvesting element may comprise a rectifier and capacitor. The non-linear impedance element may be a bipolar transistor, a field-effect transistor or a voltage controlled oscillator. The time-varying element may be a switch or a mixer. The mixer may be operably connected to receive (i) an input from a voltage controlled oscillator, and (ii) an input driven by induced voltage from the carrier signal.

In another embodiment, there is provided a reader device comprising (a) a signal source; (b) a wave signal generator; (c) a wave signal detector; and (d) a signal processing unit. The device may further comprise a filtering circuit, such as a high pass filter, a notch filter or a resonant LC tank. The device may further comprise an analog to digital converter. The signal processing unit may employ a finite impulse response-matched filter, or a frequency domain method such as a short-time Fourier Transform, a windowed fast Fourier Transform, or a spectrogram. The signal processing unit may employ a look-up table.

Also provided are systems for transmitting information from a sensor comprising the transmitter tag device and the reader device described above. Still another embodiment is a method for transmitting information from the transmitter tag to the reader in the aforementioned system, the method comprising transmitting a carrier signal from the reader device; wherein the carrier signal (a) is received by the transmitter tag and energizes the tag; (b) is altered by the tag to introduce out of band electromotive force (emf) orthogonal to the carrier signal; (c) is then modified by the non-linear impedance element connected to the sensor that measures a condition in an environment in which the transmitter tag is located; (d) is returned to the reader where the carrier signal is filtered from the out of band emf; and (e) is analyzed by the signal processing device.

Specific exemplary embodiments may have applications for children with increased fluid causing pressure inside their heads and shunts that drained the extra fluid out to keep the pressure down. This is a common problem (1-2 children per 1,000). These children may have their shunts malfunction every few years. Early symptoms are nonspecific and common to a lot of other conditions (headache, vomiting, decreased activity). Therefore, these children can be required to travel by ambulance to an institution with neurosurgical capability to have their shunts evaluated every time they get a cold or the stomach flu. Part of this evaluation involves a CT scan of their heads, which is a lot of radiation for a young child. Even one CT scan is enough radiation to impair skull and brain growth. It is not clear what this does to the child's cancer risk, but some sources estimate that for every 2,000 head CTs performed on an adult, one will die of fatal thyroid cancer. If this data is taken as a starting point, and then you consider that the thyroid of a child is much closer to the brain, gets a higher radiation dose per gram of tissue, and children presumably will live longer and be more likely to manifest their cancer, then it becomes clear that this could be a harmful practice. Currently the medical costs for hydrocephalus are over $1 billion per year.

Exemplary embodiments disclosed herein would allow a user to place a pressure monitor in the tip of the shunt to monitor pressure inside of the head. In specific embodiments, this information would be transmitted wirelessly to the RFID reader and obviate the need for long and expensive ambulance transfers, subspecialty consultation, and the use of harmful radiation. With this novel signal transduction technique, it is believed that the need for frequent calibration and the problem of data drift will be finally solved. This technology could literally transform the care of this subset of patients.

Additional benefits may also be realized. For example, if one were to use the transducer disclosed herein to monitor blood sugars, it can be paired with a wearable insulin pump to monitor and treat blood sugars in diabetics. The 25.8 million people in the United States with diabetes would never need to check their blood sugars again. Blood sugar would be wirelessly monitored with this technology and could be automatically corrected. The vastly improved signal-to-noise ratio of this novel transduction technique makes this possible.

In addition to being used to measure pressure or blood glucose, exemplary embodiments of this technology can be employed to measure pH or tissue oxygen saturation, improving the diagnosis and treatment of people with sepsis or heart failure. It can provide instant feedback on the response to therapy for these critically ill people and improve their short-term and long-term outcomes.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

These and other objects, features, and advantages of the invention will become apparent from the detailed description below and the accompanying drawings.

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1—Traditional back-scatter modulation used in RFID systems. Blue line shows unmodulated carrier, red line shows the effect of back-emf as a small change in amplitude.

FIG. 2—Unmodulated carrier signal (blue) and carrier signal with a small back-emf in the third harmonic (red).

FIG. 3—FFT of carrier signal without back-emf (blue) and with back-emf (red) for the waveforms in FIG. 2 where the back-emfs appear in the third harmonic.

FIG. 4—Prototype passive transmitter design.

FIG. 5—Prototype receiver design.

FIG. 6—Reader side of the device.

FIG. 7—Tag side of the device.

FIG. 8—Plots indicating power across the frequency spectrum for different pressure levels.

FIG. 9—Power versus pressure graphs for each of the first 6 harmonics (not including carrier).

### DETAILED DESCRIPTION OF THE INVENTION

### A. General Overview of the Invention

Current passive RFID devices have a reader coil and a tag coil that communicate and transfer data through a technique commonly called backscatter modulation. When the reader is in the vicinity of the tag, the electromagnetic signal transmitted by the reader coil (antenna) powers up the tag coil (antenna). The tag uses the same signal to send back information to the reader by modulating the reader carrier signal, usually by causing a small change in the amplitude of the carrier signal. The effect of the modulation on the reader coil is typically very small (usually 50 to 100 times weaker) as compared to the carrier wave, and therefore requires a highly sensitive detector.

The present invention involves a method for inducing a back electromotive force (emf) waveform that has most of its energy in the signal space orthogonal to the carrier waveform. In specific embodiments, there are provided both non-linear and time-varying controllable circuit elements that generate such a back emf, as well as optimal signal processing methods for detecting the data signal embedded in the back emf. These designs involve a radical change from amplitude modulation to frequency shifting of the backscattered signal. The concept is to use a controlled, non-linear impedance or time-varying element produce the required orthogonal signal components in the back emf. The impedance of the non-linear device is controlled by the sensor in context. Due to the variation in the impedance of the non-linear device based on the sensor output, the non-linear device produces harmonics whose amplitude and phase vary as a function of the sensor output. Thus, when the reader receives the tag signal, the reader carrier signal and the tag signal are orthogonal to each other in the Fourier domain. This makes it easier for the reader to distinguish between what it sent to the tag and what the tag sent it back. The reader is connected to a Signal Processing unit that optimally filters the sensed signal or the tag signal received by the reader to estimate the sensor output with the optimum signal to noise ratio. While this design potentially has applications to a variety of short-range communication problems, it is especially of interest to medical implants, where the simplicity and sensitivity of the invention can enable very low-cost, small form-factor passive devices.

This subject matter is in part motivated by the need for applications that involve monitoring a slowly varying sensor. While this design potentially has applications to a variety of short-range sensing problems, it is especially of interest to medical implant applications, where the simplicity and sensitivity of the invention can enable very low-cost, small form-factor passive devices. An example of such an application is an implanted device monitoring signals such as blood sugar, pressure, etc. The reader's goal in such an application is to obtain a readout of a small number of slowly-varying physical variables each of which is transduced into analog voltages.

One concept is to use a controlled, non-linear impedance or time-varying element whose harmonics produce the required frequency shifting on the tag signal. The impedance of the non-linear device is controlled by the sensor in context. Due to the variation in the impedance based on the sensor output, the non-linear device produces harmonics whose amplitude and phase vary as a function of the sensor output. Thus when the reader receives the tag signal, the reader carrier signal and the tag signal are orthogonal to each other in the Fourier domain. This makes it easier for the reader to distinguish between what it sent to the tag and what the tag sent it back.

The reader is connected to a signal processing unit that optimally filters the sensed signal or the tag signal received by the reader to estimate the sensor output with the optimum signal to noise ratio. One possible embodiment of an optimal filter is a device that samples and digitizes the induced signal, performs a Fast Fourier Transform on the sampled signal, records the amplitude and phase of the Fourier coefficients corresponding to the dominant harmonics in the induced signal, i.e., frequencies that are small multiples of the carrier signal frequency. The sensor variables to be read-out are then calculated as polynomial functions of the harmonic coefficients, the polynomials themselves to be determined using a one-time calibration process.

These and other aspects of the invention are described in greater detail below.

### B. Waveform Signal Transduction

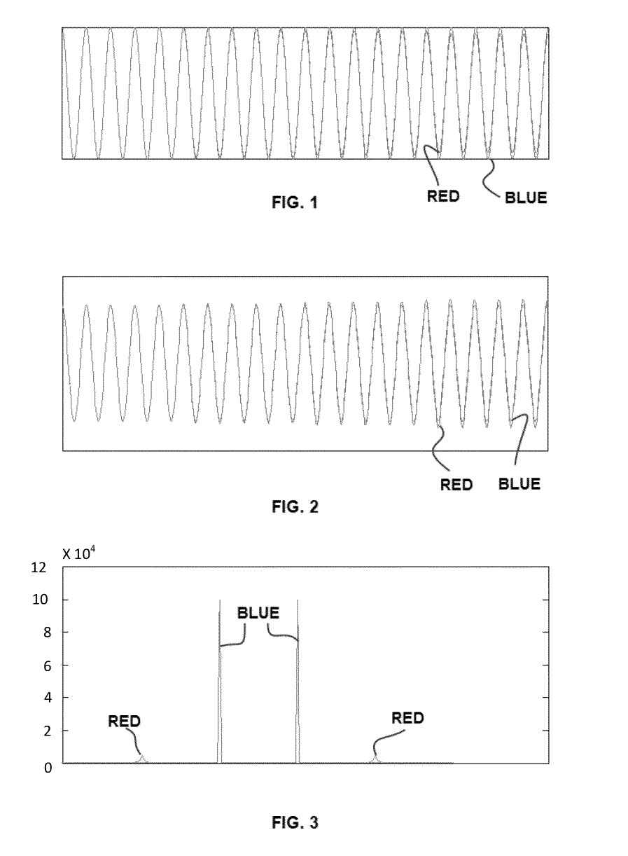

The concept behind the present invention is illustrated using a set of simulations shown in FIGS. 1-3. As shown in FIG. 1, the back emf signal is usually much weaker (typically even weaker than what is shown in the simulation above) than the carrier signal. This requires that the detection circuit at the receiver must be highly sensitive and finely calibrated in order to accurately detect the amplitude variations.

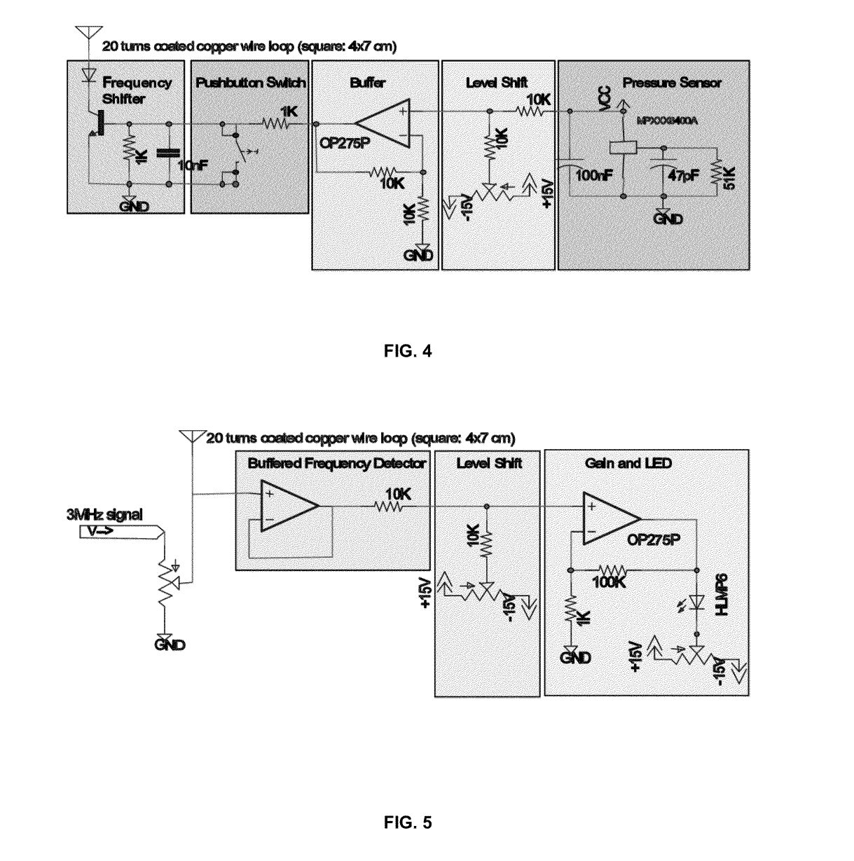

FIG. 2, on the other hand shows the unmodulated carrier along with a back-emf. The amplitude of the back-emf in FIG. 2 is the same as that of FIG. 1; however, in FIG. 2, the back-emf signal appears in the third harmonic of the carrier signal.

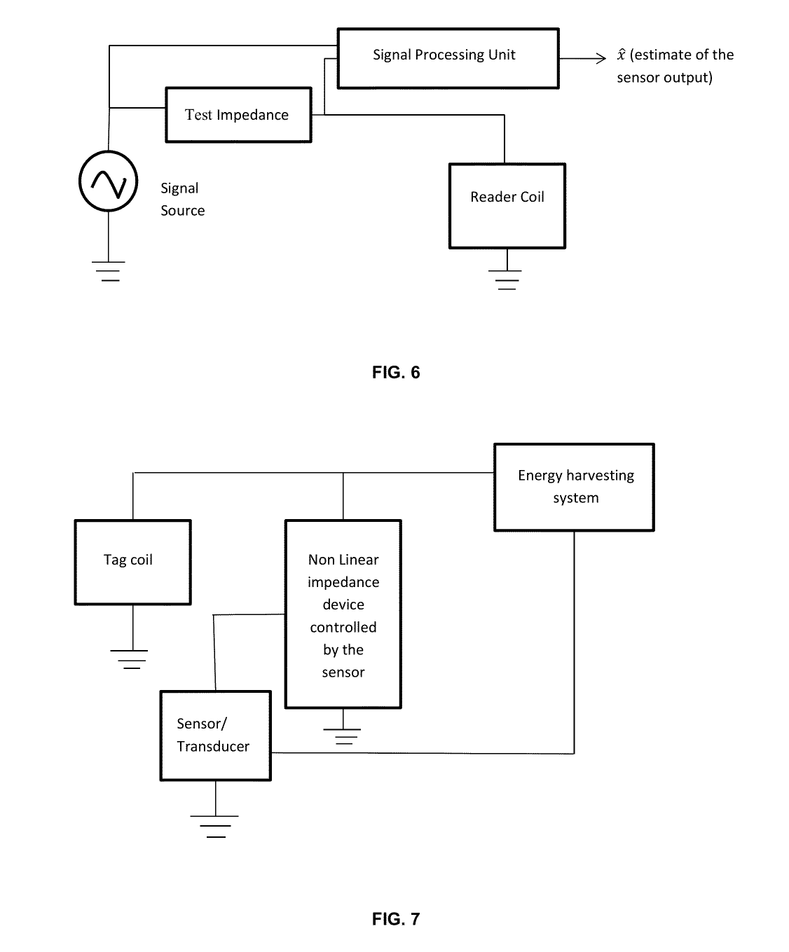

FIG. 3 shows the FFT of the carrier signal and the back-emf. The third harmonic is clearly visible, and even though its amplitude is significantly smaller than the fundamental carrier, the sensitivity of the FFT detector is far superior to that of the envelope detector.

### C. Reader-Tag System

In its most general form, the invention applies to a passive RFID system where a reader coil queries a tag coil by transmitting a sinusoidal carrier signal, which induces a current in the tag coil by inductive coupling. The design has the following elements:
a tag coil circuit has a nonlinear and/or time-varying controllable impedance which varies according to the value of a sensor (which is the device that the RFID reader is ultimately trying to “read”); controllable impedance causes out-of-band currents in the tag coil which then induce corresponding “out-of-band” back-emfs in the reader coil (“out-of-band” means that the induced back-emf has signal components that are orthogonal to the carrier signal waveform);
a signal processing circuit in the reader coil optimally filters the back-emfs so that the large carrier signal itself is suppressed leaving only the portions of the back-emf that fall in an orthogonal signal space to the carrier signal; for instance, a frequency-selective filter may remove the fundamental frequency of the carrier signal leaving behind only the harmonics
   Unlike the fundamental frequency component which is dominated by the large carrier signal, the harmonics are entirely due to the controllable impedance. Specifically in the absence of a tag, there are no harmonics. This leads to a significant increase in the sensitivity of the RFID system, as instead of detecting a very small variation in a large signal, one is instead looking for a signal in an unoccupied part of the signal space. This increased sensitivity is an important advance of the present invention.

This design can be used to “read” an analog sensor, but can also be used to send digital data by using standard modulations such as On/Off Keying (OOK), Frequency Shift Keying (FSK), Amplitude Shift Keying (ASK) and Phase Shift Keying (PSK). This design is applicable to both passive tags and active tags that have a power source such as a battery. As pointed out earlier, the passive version of this invention is especially well-suited to accurate detection of slow-varying analog variables. Some specific embodiments are listed below:
a voltage controlled oscillator as the non-linear circuit element; in this case, the back-emf waveform is completely independent of the carrier signal, whose only purpose is to energize the tag if it is passive;
a mixer and voltage controlled oscillator (VCO) as the non-linear and time-varying circuit element; in this case, the sensor voltage to be read changes the frequency of a sinusoidal signal produced by a VCO which drives one of the inputs of a mixer, the other input of the mixer being driven by the induced voltage from the carrier signal, equivalent to a pure frequency-modulation;
a power law device, which acts like a non-linear voltage controlled impedance (simple example of such is a field-effect transistor).
   The signal processing operations to implement an optimal detector will in general depend on the type of the non-linear or time-varying device employed in the embodiment. Specific examples of such signal processing operations include one or more of the following operations (a) converting back-emf waveform into the frequency domain using tools such as the FFT, spectrogram or short-time Fourier Transform, and nulling out the frequency components corresponding to the carrier signal, (b) a matched filter implemented as a FIR or IIR filter in the time-domain or in the frequency domain, and (c) a look-up table showing the mapping of sensor output values to the magnitude and phase of different frequency components.

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, SRAM, NVRAM, EEPROM, FLASH memory, magnetic data storage media, optical data storage media, or the like. The instructions may be executed to support one or more aspects of the functionality described in this disclosure. Various examples have been described. These and other examples are within the scope of the following claims.

### D. Device Components and Construction and Applications

Implantable medical devices (IMDs) have been used for therapeutic and functional restoration indications for animals, including humans, for a number of years. These IMD housing must be constructed in such a way that produces a hermetically sealed housing or case and of a material that is biocompatible. Ceramics, epoxies, and metals, such as titanium or titanium alloys have been the mainstay for many IMDs, including implantable pulse generators, pacemakers, and drug delivery pumps, for example. Another class of material, known as thermoplastic liquid crystal polymers (LCP) have a unique combination of properties that make it well suited for encasing IMDs. LCP is extremely inert in biological environments and has barrier properties an order of magnitude greater than epoxy plastic materials and is virtually impermeable to moisture, oxygen, and other gases and liquids.

The implantable wireless platform may also need an anchoring mechanism in order to avoid movement of the implant components. Anchoring provisions may be incorporated directly into the platform (for example part of the housing) or may alternatively be added with an additional assembly step. An example of this would be insertion of the implantable part of the platform into a molded plastic or metal shell that incorporates anchoring provisions. Many such packaging schemes are known to those familiar with the art, and the present description should not be construed as limiting.

The anchoring mechanism can be any type of anchor known in the art, for example the implantable unit can be attached to the skull or scalp using wires, screws (helical or otherwise), bolts, a mesh, stents, springs, stitches, a tine that expands, etc. The anchoring mechanism can also be part of another device. The anchoring mechanisms can be made from one or more of the following materials, but not limited to, Nitinol, Teflon, Parylene, Polymer, or Metal.

Intracranial pressure can increase as a result of both illness and injury, including traumatic brain injury (TBI), brain tumors, and hydrocephalus. Current devices for monitoring ICP have several limitations, including wires that can limit patient mobility and baseline drift (signal instability). The present invention would permit implantation of a device to allow for safer and easier patient transport, as well as more streamlined device calibration and accuracy. An example of a wireless pressure sensor for implantable use is described in U.S. Patent Publication 20090299216.

Other types of physiologic conditions that can be monitored in accordance with the present invention include blood pH, blood pressure, blood gas levels, blood glucose levels, blood alcohol levels, blood drug levels (including controlled substances), blood metabolite levels, renal output, cardiac output, cardiac contraction period or force, temperature, or any other medically relevant parameter.

Non-medical uses include for any environmental measurement, such as soil moisture sensors buried underground, water flow sensors located underwater, structural monitoring sensors, such as in buildings that measure stress and movement, identification tags on goods, for use in monitoring transit and final location, and for forensic purposes.

In the case of a temperature probe, one exemplary temperature probe comprises two probe leads connected to each other through a temperature-dependent element that is formed using a material with a temperature-dependent characteristic. An example of a suitable temperature-dependent characteristic is the resistance of the temperature-dependent element. The two probe leads comprise, for example, a metal, an alloy, a semimetal, such as graphite, a degenerate or highly doped semiconductor, or a small-band gap semiconductor. Examples of suitable materials include gold, silver, ruthenium oxide, titanium nitride, titanium dioxide, indium doped tin oxide, tin doped indium oxide, or graphite. The temperature-dependent element can further comprise a fine trace (e.g., a conductive trace that has a smaller cross-section than that of the probe leads) of the same conductive material as the probe leads, or another material such as a carbon ink, a carbon fiber, or platinum, which has a temperature-dependent characteristic, such as resistance, that provides a temperature-dependent signal when a voltage source is attached to the two probe leads of the temperature probe. The temperature-dependent characteristic of the temperature-dependent element can either increase or decrease with temperature.

The sensor element may be manufactured from biocompatible materials, such as materials that are corrosion resistant, including Pt, SiO₂ coatings, and glass thin films. In addition, corrosion resistant materials that are harmless to tissues in biologic environments, such as silicon and heavily boron-doped silicon can be used in the manufacture of the components of the internal unit. Another method by which the corrosion resistance of the internal unit can be improved is through coating of the internal unit with titanium, iridium, Parylene (a biocompatible polymer), or various other common and/or proprietary thick and thin films.

The sensor optionally comprises a biocompatible coating. The bioactive polymers are in general biocompatible, i.e., physiologically tolerated, and do not cause substantial adverse local or systemic responses. While synthetic polymers such as poly(tetrafluoroethylene), silicones, poly(acrylate), poly(methacrylate), hydrogels, and derivatives thereof are most commonly used, natural polymers such as proteins and carbohydrates are also suitable. The bioactive polymer layer functions to protect the implant, preserve its function, minimize protein adsorption onto the implant, and serve as a site for the delivery of the tissue response modifying agents and drugs as well as other drugs and factors.

### E. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

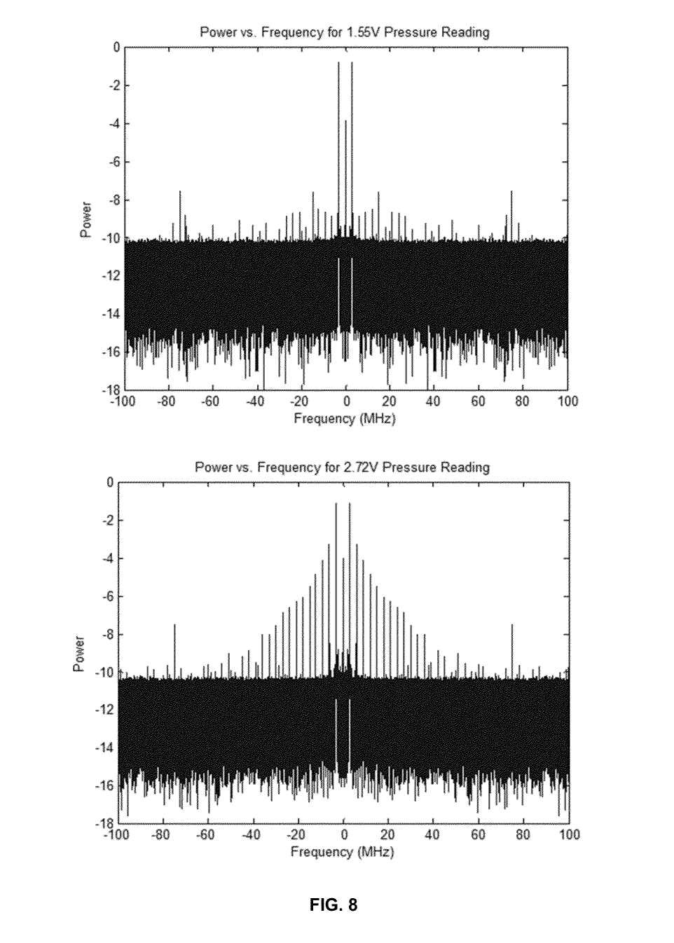

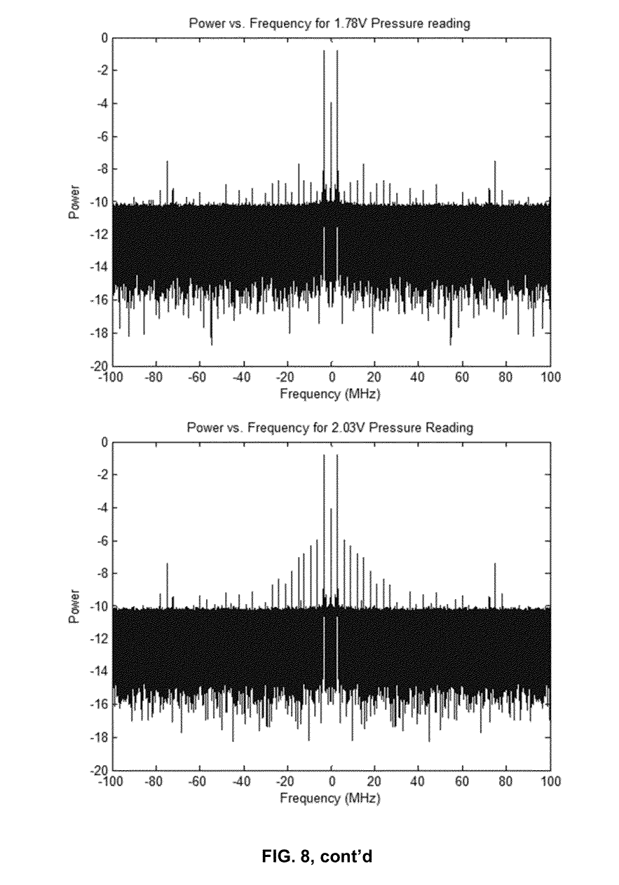

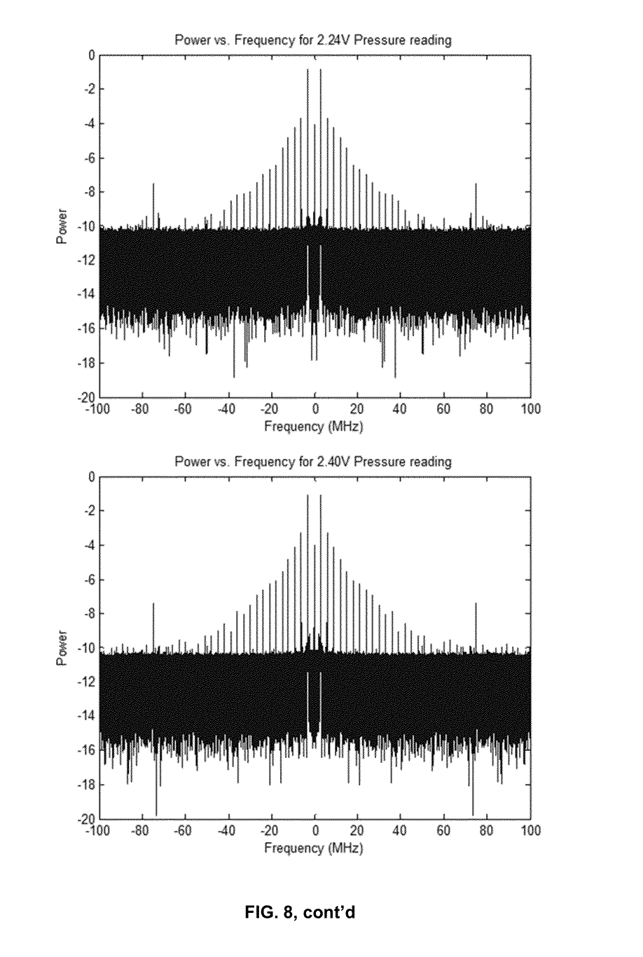

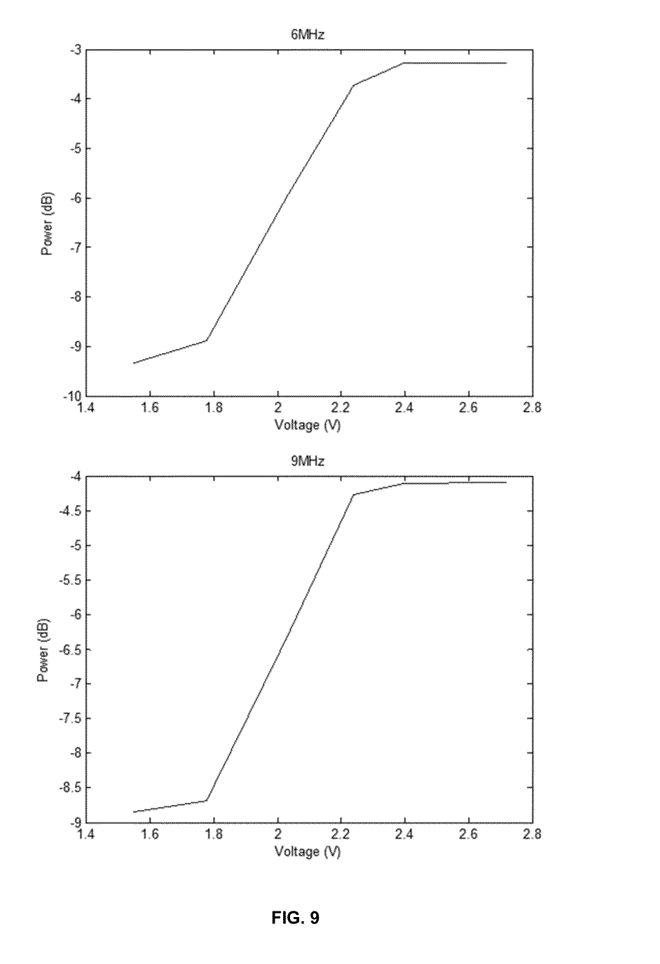

FIGS. 4-5 are circuit diagrams of reader and tag corresponding to one particular simple embodiment of the invention that uses a simple bipolar transistor as the non-linear impedance and an envelope detector as the detection device. FIGS. 6-7 are general schematics of the system.

One contemplated design exploits the frequency-dependence of the open-loop gain and common-mode rejection ratio (CMRR) of an inexpensive, low-end operational amplifier. It can be shown (Philips, 1988) that the DC gain of a voltage follower buffer is given by:

$$\frac{A ( 2 C M R R + 1 )}{2 C M R R ( 1 + A ) - A}$$

where A is the open loop gain of the op amp. The frequency-dependence of this A and CMRR results in an offset voltage that varies by nearly 2 volts between 1 MHz and 10 MHz. This varying offset voltage can be used to detect the presence and strength of the harmonics which are introduced by the sensor.

### Example 2

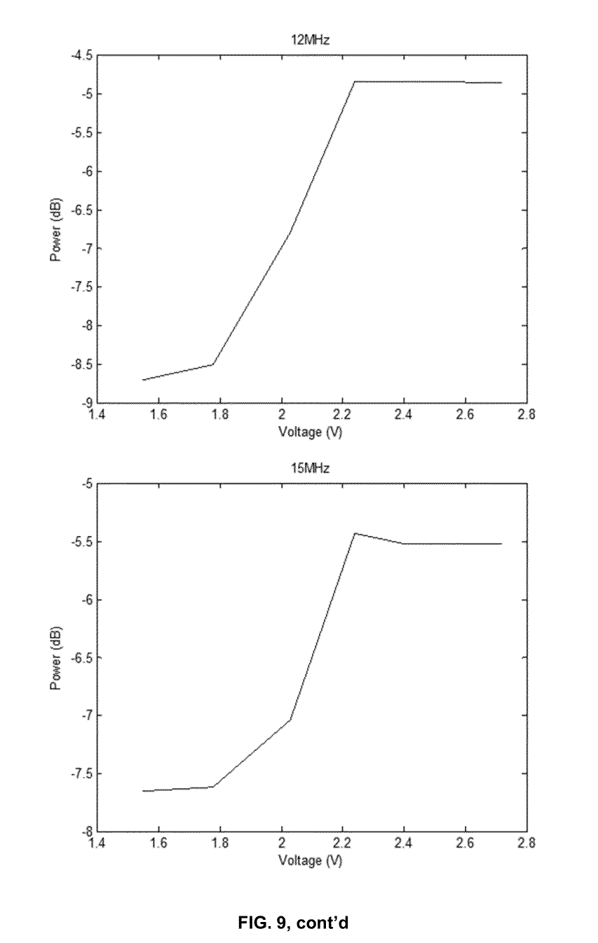

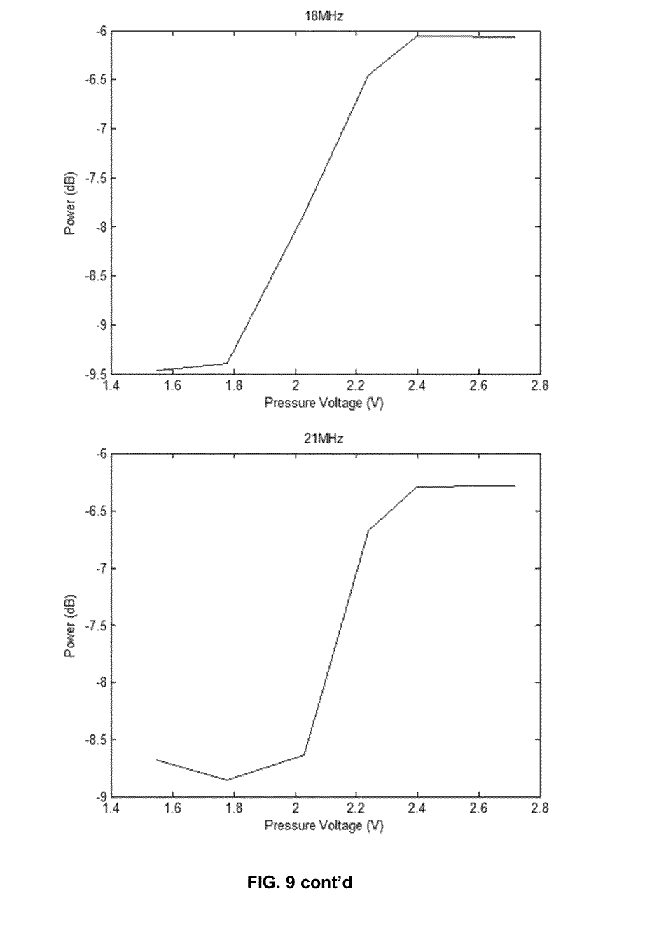

A reader circuit has a carrier frequency of 3 MHz, and induces a 3 MHz sinusoid on the sender circuit. Pressure Sensor voltage output introduces harmonics of the carrier frequency on the sender circuit. The goal is to determine the pressure based on the induced waveform in the reader circuits. To determine this we looked at the induced frequency spectrum to determine a possible relation between different power levels in each harmonic and pressure. The plots in FIG. 8 indicate power across the frequency spectrum for different pressure levels. The inventors then examined power versus pressure for each of the first 6 harmonics (not including carrier), as shown in FIG. 9.

To determine a model for this, the inventors tried a few simple functions to fit:

V=a0+a1*Pᵢ+a2*Pᵢ²+a3*Pᵢ

V=a0+a1*Pᵢ+a2*Pᵢ*Pⱼ+a3*Pⱼ

V=a0+a1*Pᵢ+a2*Pⱼ+a3*Pₖ

V is the sensed signal (pressure sensor voltage), a0, a1, a2 and a3 are constants and P, is the power in the i^{th} harmonic. To fit these models, they used method of least squares to calculate a0, a1, a2 and a3 and then calculated which model and pressure combinations had the least squared error from the original data set:

TABLE 1

| | | | | Power | Power | Power |
|---|---|---|---|---|---|---|
| | Power | Power | Power | (15 | (18 | (21 |
| Voltage | (6 MHz) | (9 MHz) | (12 MHz) | MHz) | MHz) | MHz) |
| 1.55 | −9.335 | −8.840 | −8.701 | −7.657 | −9.463 | −8.682 |
| 1.78 | −8.886 | −8.680 | −8.501 | −7.621 | −9.394 | −8.858 |
| 2.03 | −5.970 | −6.329 | −6.801 | −7.039 | −7.864 | −8.634 |
| 2.24 | −3.720 | −4.268 | −4.846 | −5.431 | −6.458 | −6.676 |
| 2.40 | −3.276 | −4.112 | −4.854 | −5.521 | −6.058 | −6.287 |
| 2.72 | −3.280 | −4.099 | −4.857 | −5.526 | −6.066 | −6.290 |

After performing the following calculations, we determined that the second model worked best for Powers in the 2^{nd} and 3^{rd} harmonics (6 MHz and 9 MHz). The resulting equation is:

V=2.4943+1.1429*P₂+0.0285*P₂*P₃−0.8339*P₃

It should be observed that while the foregoing detailed description of various embodiments of the present invention is set forth in some detail, the invention is not limited to those details and an implantable neurostimulator or neurological disorder detection device made according to the invention can differ from the disclosed embodiments in numerous ways. In particular, it will be appreciated that embodiments of the present invention may be employed in many different applications to detect anomalous neurological characteristics in at least one portion of a patient's brain. It will be appreciated that the functions disclosed herein as being performed by hardware and software, respectively, may be performed differently in an alternative embodiment. It should be further noted that functional distinctions are made above for purposes of explanation and clarity; structural distinctions in a system or method according to the invention may not be drawn along the same boundaries. Hence, the appropriate scope hereof is deemed to be in accordance with the claims as set forth below.

### F. References

The following references, to the extent that they provide exemplary procedural or other details supplementary to those set forth herein, are specifically incorporated herein by reference.
[1] Tofighi, M.-R.; Kawoos, U.; Neff, S.; Rosen, A.;, “Wireless intracranial pressure monitoring through scalp at microwave frequencies,” Electronics Letters, vol. 42, no. 3, pp. 148-150, 2 Feb. 2006.
[2] Foster, K. R.; Jaeger, J.;, “RFID Inside,” Spectrum, IEEE, vol. 44, no. 3, pp. 24-29, March 2007.
[3] world-wide-web at google.com/patents/US6997388
[4] Ativanichayaphong, T.; Shou-Jiang Tang; Lun-Chen Hsu; Wen-Ding Huang; Young-Sik Seo; Tibbals, H. F.; Spechler, S.; Chiao, J.-C.;, “An implantable batteryless wireless impedance sensor for gastroesophageal reflux diagnosis,” Microwave Symposium Digest (MTT), 2010 IEEE MTT-S International, vol., no., pp. 608-611, 23-28 May 2010.
[5] world-wide-web at google.com/patents/US20030017804?dq=rfid+tag&ei=jYI_UOTqLsfkqgGPg4H4BA.
[6] world-wide-web at springerlink.com/content/xhq7581v57xmg836/fulltext.html.
[7] thejns.org/doi/abs/10.3171/foc.2007.22.4.12.
[8] online.liebertpub.com/doi/pdf/10.1089/neu.2009.0951.
[9] ieeexplore.ieee.org/stamp/stamp.jsp?tp=&arnumber=6090758.
H. C. Gomes and N. B. Carvalho, “The use of intermodulation distortion for the design of passive RFID,” pp. 377-380, 2007.
Philips, “Integrated operational amplifier theory,” AN165 application notes, November 1988.

What is claimed is:

1. A passive transmitter tag device suitable for implantation into a subject, said device comprising:
(a) a non-linear impedance element or time-varying element for introducing harmonics or frequency-shifted signal components into a back-emf produced by a sinusoidal carrier signal;
(b) an energy harnessing element;
(c) sensor/transducer for monitoring a patient condition, wherein said sensor is operably connected to said non-linear impedance element or time-varying element.

2. The device of claim 1, wherein said sensor/transducer is a pressure monitor.

3. The device of claim 1, wherein said sensor/transducer is a pH monitor.

4. The device of claim 1, wherein said sensor/transducer is a temperature monitor.

5. The device of claim 1, wherein said sensor/transducer is a blood glucose monitor.

6. The device of claim 1, wherein the energy harvesting element is a rectifier and capacitor.

7. The device of claim 1, wherein the non-linear impedance element is a bipolar transistor, a field-effect transistor or a voltage controlled oscillator.

8. The device of claim 1, wherein the time-varying element is a switch.

9. The device of claim 1, wherein the time-varying element is a mixer.

10. The device of claim 9, wherein the mixer is operably connected to receive (i) an input from a voltage controlled oscillator, and (ii) an input driven by induced voltage from the carrier signal.

11. A reader device comprising:
(a) a signal source;
(b) a wave signal generator;
(c) a wave signal detector; and
(d) a signal processing unit.

12. The device of claim 11, further comprising a filtering circuit.

13. The device of claim 12, wherein said said filtering circuit is a high pass filter, a notch filter or a resonant LC tank.

14. The device of claim 11, further comprising an analog to digital converter.

15. The device of claim 11, wherein the signal processing unit employs a finite impulse response-matched filter.

16. The device of claim 11, wherein the signal processing unit employs a frequency domain method.

17. The device of claim 16, wherein the frequency domain method is a short-time Fourier Transform, or a windowed fast Fourier Transform.

18. The device of claim 16, wherein the frequency domain method is a spectrogram.

19. The device of claim 11, wherein the signal processing unit employs a look-up table.

20. A system for transmitting information from a sensor comprising (a) the transmitter tag device of claim 1 and (b) the reader device of claim 11.

21. A method for transmitting information from the transmitter tag to the reader in the system of claim 20, the method comprising transmitting a carrier signal from the reader device; wherein the carrier signal:
(a) is received by the transmitter tag and energizes the tag;
(b) is altered by the tag to introduce out of band electromotive force (emf) orthogonal to the carrier signal;
(c) is then modified by the non-linear impedance element connected to the sensor that measures a condition in an environment in which the transmitter tag is located;
(d) is returned to the reader where the carrier signal is filtered from the out of band emf; and
(e) is analyzed by the signal processing device.
